(19) United States
(12) Patent Application Publication
(10) Pub. No.: US 2011/0104775 A1
(43) Pub. Date: May 5, 2011

(54) Method to Increase the Ethanol Concentration from the Conversion of Lignocellulose

(75) Inventors: Teng-Chieh Hsu, Taitung County (TW); Ting-Hsiang Lin, Taoyuan County (TW); Tien-Yang Ma, Taoyuan County (TW); Gia-Luen Guo, Taipei County (TW); Wen-Song Hwang, Taoyuan County (TW); Jia-Baau Wang, Taipei (TW)

(73) Assignee: ATOMIC ENERGY COUNCIL-INSTITUTE OF NUCLEAR ENERGY RESEARCH, Taoyuan County (TW)

(21) Appl. No.: 12/856,073

(22) Filed: Aug. 13, 2010

(30) Foreign Application Priority Data
Oct. 30, 2009 (TW) 098136802

(51) Int. Cl. C12P 7/10

(52) U.S. Cl. 435/165

(57) ABSTRACT

The present disclosure is related to a method for increasing the ethanol concentration from the conversion of lignocellulose. The pretreated solid residues are mixed with ethanol-containing broth from the fermentation of xylose hydrolysate by Pichia stipitis and then are performed under the process of simultaneous saccharification and fermentation (SSF) with Sacharomyces cerevisiae and cellulase for converting cellulose to ethanol. The final ethanol concentration in broth as well as the ethanol productivity is increased at least 1.8 times in comparison of conventional process for lignocellulosic ethanol production.

### CROSS REFERENCE TO RELATED PATENT APPLICATIONS

This application claims priority from Taiwan Patent Application No. 098136802, filed in the Taiwan Patent Office on Oct. 30, 2009, and incorporates the Taiwan patent application in its entirety by reference.

### TECHNICAL FIELD OF THE DISCLOSURE

This present disclosure relates to increase the ethanol concentration from the conversion the lignocelluloses. In particular, it related to a designed process for integrating the C5 sugar fermentation and the cellulose-to-ethanol process under an operative mode of simultaneous saccharification and fermentation process (SSF). It is efficient for increasing the final ethanol concentration in the broth of fermentor.

### DESCRIPTION OF THE RELATED ARTS

Increasing the consumption of petroleum has results in the necessity for developing novel liquid biofuel. Among them, the Bioethanol has been considered as one of the most significant liquid biofuel, which could be blend with gasoline to reduce the use of fossil fuel and then decrease the emission of CO₂ and air pollutants.

Today, bioethanol is almost produced via fermentation process. According to the source of feedstock, the bioethanol is classified starch-based, cane sugar-based, and cellulosic ethanol. The feedstock of starch-based and cane sugar-based ethanol is mainly obtained from grain and sugarcane. The cellulosic ethanol is made from composition of cellulose and hemicelluloses in plant and agricultural waste. It is important to note that bioethanol from grain or sugarcane is usually considered to be very practicable and the process could give high ethanol concentration in broth due high ethanol concentration produced. However, the lignocellulosic materials have shown the advantages of low cost, diverse and no competition with food crops. Consequently, lignocellulosic material is potentially considered as the feedstock for bioethanol production.

The cellulosic ethanol process is related to a heterogeneous reaction between solid and liquid phase. If increasing the solid content in process, the high solid content often make the delivery and agitation of pretreated solid residues difficult. The poor substrate fluidity usually results in the mass transfer limitation, and then causes the conversion efficiency to be decreased as well as the sugar concentration produced in process. Therefore, the cellulosic ethanol concentration from the broth of fermentation process is generally lower than that in cane sugar-based and starch-based process. When the broth with low ethanol concentration is purified to hydrous ethanol concentration by distillation, the energy consumption of distillation will be greatly increased. Thus, the ethanol concentration in broth is generally proposed to be above 4% for reducing the energy consumption of ethanol distillation.

The lignocellulosic feedstock is mainly composed of 60%˜80% cellulose and hemicelluloses and 15-25% lignin. The hemicelluloses first converted to pentose by pretreatment process, and then the mono-sugar released could be fermented into ethanol. For pretreatment technologies of hemicelluloses hydrolysis, the thermal chemical pretreatment such as dilute acid hydrolysis and acid-catalyzed steam hydrolysis is usually used in cellulosic ethanol process. During these pretreatment technologies, constant solid and liquid content is mixed with 1˜3% (w/w) diluted sulfuric acid under high temperature and pressure conditions. The liquid is called hydrolysate after pretreatment reaction. The fermentation inhibitors such as furfural, hydroxylmethyl furfural (HMF) or organic acid is produced along as the release of xylose. These inhibitors often show toxic and inhibitive to fermented organism and further reduce the ethanol productivity. Therefore, the furfural of hydrolysate from pretreatment reaction is often removed by overliming process. Then the detoxified hydrolysate is available to be fermented by organism.

After the pretreatment, the solid residues which contain long chain cellulose is needed to be hydrolyzed to monosugar (C₆H₁₀O₅)n+nH₂O→nC₆H₁₂O₆) by dilute acid, concentrated acid or enzyme. Now, usage of celluase for hydrolysis of cellulose is a major tendency. Traditionally, the biochemical process of cellulose-to-ethanol could be: (1) separate/sequential hydrolysis and fermentation (SHF), as shown in FIG. 6; (2) simultaneous saccharification and fermentation, as shown in FIG. 7. After separating solid and liquid which was from lignocellulosic materials dealt with dilute acid method, the xylose-containing hydrolysate is directly fermented. The solid residues are hydrolyzed by cellulase and then are fermented. In general, the process of cellulose-to-ethanol is parallel with the xylose fermentation. The ethanol concentration from glucose fermentation is higher than that from xylose fermentation. However, mixing the both ethanol broth resulted in a reduction of final ethanol concentration, which results in a need for increasing the distillation energy. Therefore, increasing the cellulosic ethanol concentration is considered as an important issue to improve the competitive for cellulosic ethanol production.

### SUMMARY OF THE DISCLOSURE

The main purpose of the present disclosure is to provide a method to increase lignocellosic ethanol concentration in broth. The pretreated solid residues are mixed with ethanol-containing broth from the fermentation of xylose hydrolysate and then performed under the process of simultaneous saccharification and fermentation (SSF) for converting cellulose to ethanol.

The second purpose of the present disclosure is to obtain a final ethanol concentration above 4% (w/w) or at least 1.8 times in comparison of that from traditional SHF process.

The third purpose of the present disclosure is to reduce 50% usage of water in the fermentation process by replacing water with ethanol broth from xylose fermentation.

To achieve the above purposes, the present disclosure is a method to increase lignocellosic ethanol concentration in broth, comprising steps of: (a) obtaining a xylose hydrolysate and a solid residue from the pretreatment of lignocellulosic materials; (b) the ethanol-containing broth is obtained from the fermentation of the xylose hydrolysate by adding Pichia Stipitis; and (c) mixing the broth from xylose fermentation with the solid residue is performed under the simultaneous saccharification and fermentation (SSF) process by adding Saccharomyces Cerevisiae and cellulase.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the flow chart for preferred embodiment according to the present disclosure;

FIG. 2 is the illustration for the variation of ethanol concentration in the xylose hydrolysate fermented with Pichia Stipitis;

FIG. 3 is the illustration for time-course of ethanol concentration under SSF process with a solid-liquid ratio of 1:10;

FIG. 4 is the illustration for time-course of ethanol concentration under SSF process with a solid-liquid ratio of 1:6.7;

FIG. 5 is the flow chart for the overliming process;

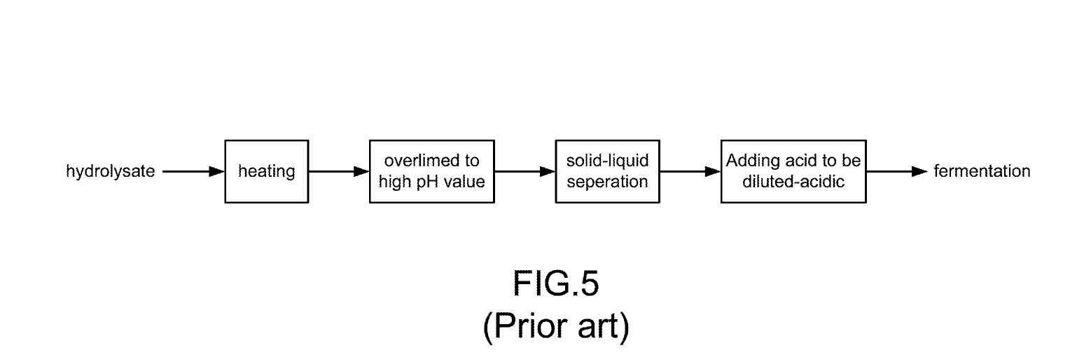

FIG. 6 is the flow chart of SHF; and

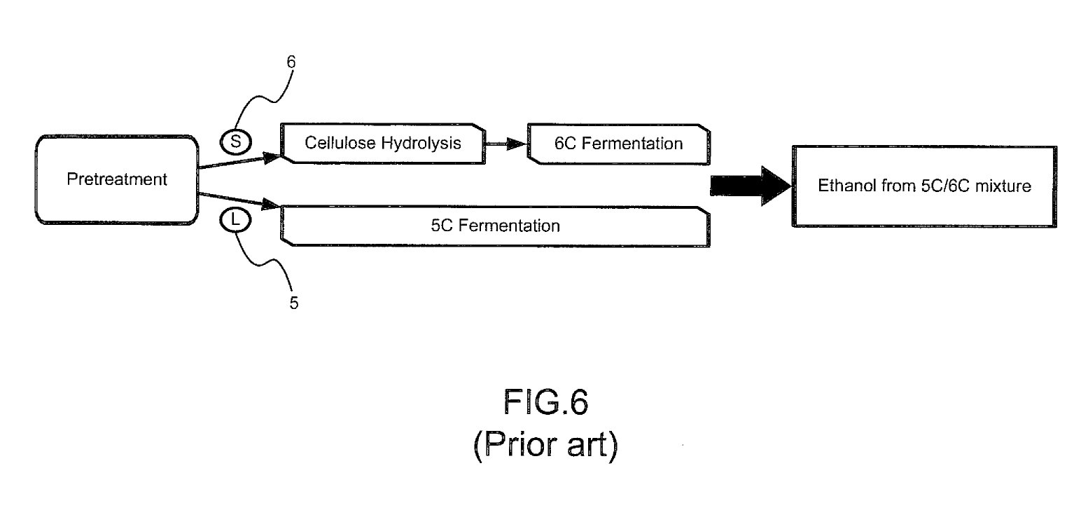

FIG. 7 is the flow chart of SSF.

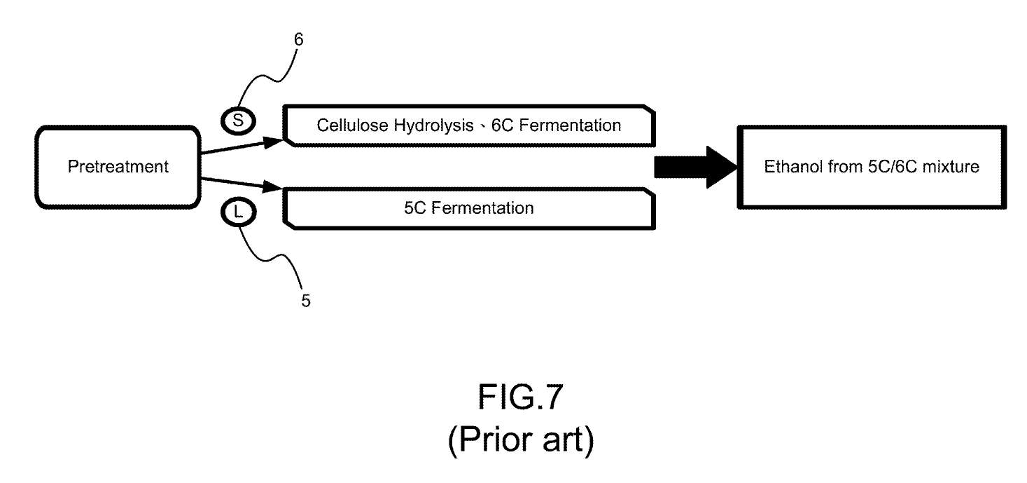

### DESCRIPTION OF THE PREFERRED EMBODIMENT

The following description of the preferred embodiment is provided to understand the features and the structures of the present disclosure.

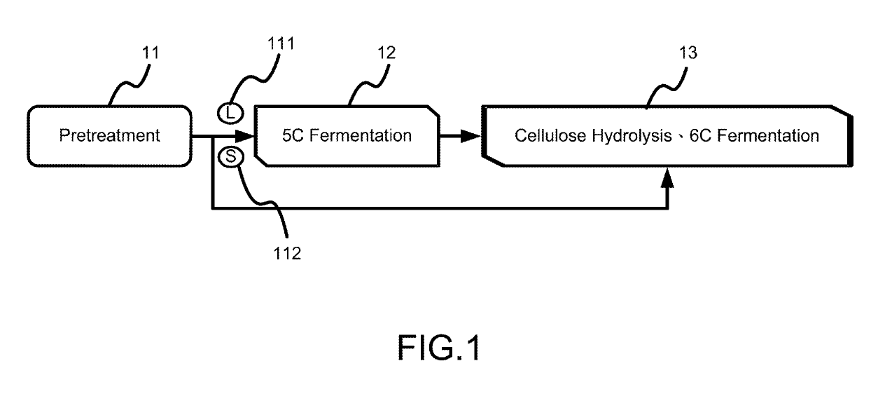

FIG. 1 is a flowchart showing a preferred embodiment according to the present disclosure. As shown in the figure, the present disclosure is a method to increase lignocellosic ethanol concentration in broth, comprising the following steps:
(a) Processing pretreatment 11: lignocellulosic materials, like rice straw, bagasse, silvergrass, napiergrass, pineapple peel, switchgrass, wood, bamboo, etc., is pretreated with acid to obtain a xylose hydrolysate and a solid residue, where the pretreatment methods can be: (1) hot water-washing by using a twin-screw extruder equipped with pressured tank; or (2) acid-catalyzed steam explosion.
(b) Xylose fermentation 12: The xylose hydrolysate after the pretreatment is directly fermented, or the xylose hydrolysate is overlimed through sequential processes, where includes heating gradually and over-adding with lime; separating liquid from solid; and adding acid to neutralize the xylose hydrolysate which has been overliming. Thus, the xylose hydrolysate finally becomes a pH value between 4.5 and 7.0.
(c) Saccharification and fermentation 13: After mixing the ethanol broth from xylose fermentation and the solid residues from pretreatment, the mixed substrate is performed under the process of simultaneous saccharification and fermentation (SSF) by adding yeast and cellulase.

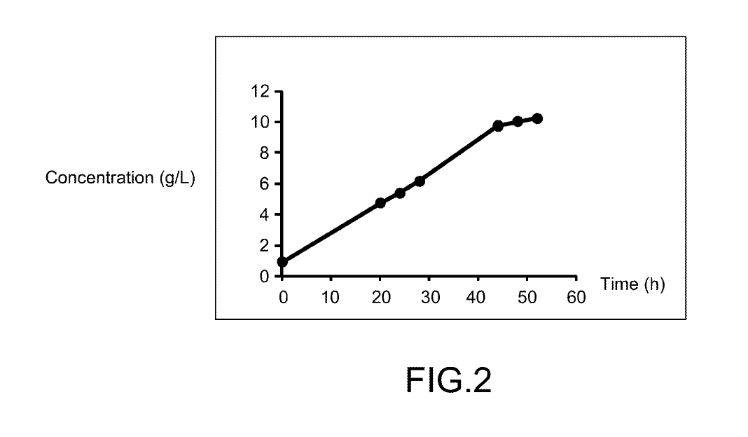

Under the present disclosure, the xylose hydrolysate and pretreted solid residuse which was used in all examples with rice straw by dilute acid steam explsion pretreatment method. The xylose hydrolysate was dealt with overliming method to remove furfural and then was fermented by Pichia stipitis in 5 L fermentor. The fermentaion was controlled in 30° C. at pH6 with 100 rpm agition. The incoulation ratio is 1:5(v/v) (incoulted volume:fermention volume). FIG. 2 is an illustration showing an ethanol concentration curve of a xylose hydrolysate fermented by Pichia Stipitis. The ethanol concentration is 10.2 g/L in 52 h after xylose fermention. The ethanol broth from xylose fermentation replaces the water to mix with the solid residues. The mixed substrates was performed under the process of simulataneous saccharification and fermentation by add cellulase and Saccharomyces cerevisiae.

### Example 1

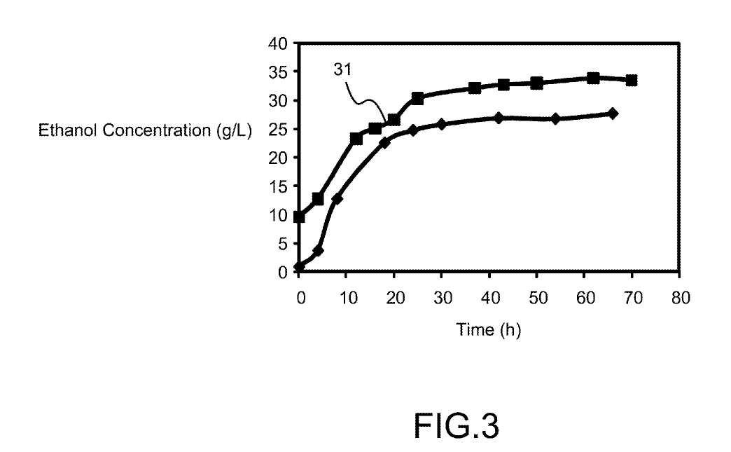

FIG. 3 is an illustration showing an ethanol concentration curve under the SSF process with a solid-liquid ratio of 1:10. This example 1 was operated in 1 L. The ethanol broth from xylose fermentation by Pichia stipitis replaced the extra water which was used in SSF process and mixed with pretreated rice straw. The pH of the mixed substrate was adjusted to 5. The Saccharoymces cereviase was added with the inoculation rate of 1:10 (v/v) (inoculation volume: fermentation volume). The SSF process was then performed at 35° C. As a result shows in an ethanol concentration curve 31, the ethanol concentration is increased from 24 g/L to 34 g/L.

### Example 2

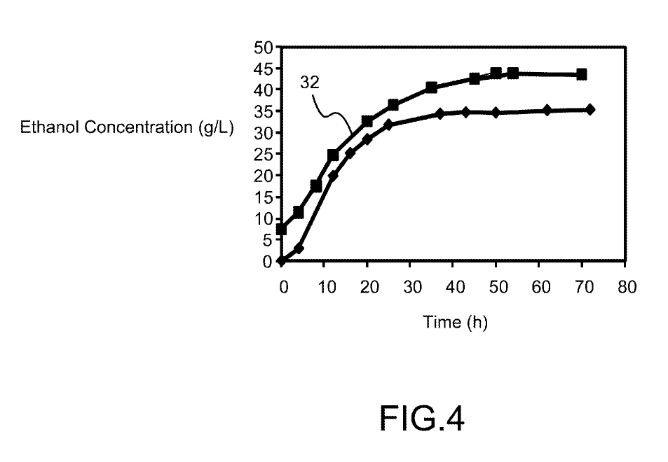

FIG. 4 is an illustration showing an ethanol concentration curve under SSF process with a solid-liquid ratio of 1:6.7. This example 2 was operated in 5 L. The ethanol broth from xylose fermentation by Pichia stipitis replaced the extra water which was used in SSF process and mixed with pretreated rice straw. The pH of the mixed substrate was adjusted to 5. The Saccharoymces cereviase was added with a inoculation ratio of 1:10 (v/v) (inoculation volume: fermentation volume). The SSF process was performed at 35° C. As a result shows in an ethanol concentration curve 32, the ethanol concentration is increased from 35 g/L to 44 g/L.

What is claimed is:

1. A method to increase the lignocellulolic ethanol concentration in broth, the method comprising:
producing xylose hydrolysate and solid residues from lignocellulosic materials by pretreatment;
obtaining ethanol-containing broth from xylose hydrolysate which is from xylose fermentation by Pichia stipitis; and
mixing the ethanol-containing broth with solid residue to obtain a mixed substrate via a simultaneous saccharification and fermentation (SSF) process by adding Saccharomyces Cerevisiae and cellulase.

2. The method according to claim 1, wherein said lignocellulosic materials are selected from a group consisting of rice straw, bagasse, silvergrass, napiergrass, pineapple peel, switchgrass, wood and bamboo.

3. The method according to claim 1, wherein said pretreatment is selected from a group consisting of (1) hotwater-washing by using a twin-screw extruder equipped with a pressured tank; and (2) acid-catalyzed steam explosion.

4. The method according to claim 1, wherein an inoculation ratio of Pichia Stipitis during xylose fermentation is between 1:5 and 1:10 (v/v).

5. The method according to claim 1, wherein said xylose fermentation is operated at a temperature between 25° C. and 30° C.

6. The method according to claim 1, wherein said xylose fermentation is processed at a pH value between 4.5 and 7.0.

7. The method according to claim 1, wherein a solid to liquid ratio of said mixed substrate is between 1:5 and 1:10 (w/w).

8. The method according to claim 1, wherein said SSF is performed at a temperature between 35° C. and 40° C.

9. The method according to claim 1, wherein an ethanol concentration above 4% (w/w) is obtained through SSF after mixing ethanol-containing broth from fermentation of xylose hydrolysate with pretreated solid residue.
